(19) United States
(12) Patent Application Publication
(10) Pub. No.: US 2001/0027269 A1
(43) Pub. Date: Oct. 4, 2001

(54) Liquid sampler and blood analyzer using the same

(76) Inventor: Yousuke Tanaka, Kobe-shi (JP)

Correspondence Address: BIRCH STEWART KOLASCH & BIRCH, PO BOX 747, FALLS CHURCH, VA, 22040-0747, US

(21) Appl. No.: 09/819,732

(22) Filed: Mar. 29, 2001

(30) Foreign Application Priority Data
Mar. 29, 2000 (JP) 2000-090413
Mar. 29, 2000 (JP) 2000-090414

(51) Int. Cl. A61B 5/00; B65D 81/00

(52) U.S. Cl. 600/368; 600/573

(57) ABSTRACT

A liquid sampler includes: a metering pump which is composed of a cylinder having opposite end openings and a cylindrical cavity, a piston inserted in the cavity from one of the openings of the cylinder, and a driving source for reciprocally and linearly moving the piston; and a pipette directly connected to the other opening of the cylinder.

### CROSS-REFERENCES TO RELATED APPLICATIONS

This application is related to Japanese Patent Applications Nos. 2000-090413 and 2000-090414, filed on Mar. 29, 2000, whose priorities are claimed under 35 USC §119, the disclosures of which are incorporated by reference in their entirety.

### BACKGROUND OF THE INVENTION

1. Field of the Invention

The present invention relates to a liquid sampler and a blood analyzer using the same and, more particularly, to a liquid sampler capable of quantitatively dispensing a very small volume of liquid such as a liquid specimen or a reagent and to a simplified blood analyzer for analyzing a blood component by mixing a blood specimen and a reagent.

2. Description of the Related Art

Automatic analyzers such as immuno-agglutination analyzers are equipped with a liquid sampler for dispensing predetermined volumes of a liquid specimen and a reagent. Recently, more accurate sampling has been required for sampling a much smaller volume.

Japanese Unexamined Patent Publication No. 10-123152 (1998), for example, discloses such arrangements that a piston of a syringe (metering member) is finely driven to maintain a liquid front at a predetermined position in a pipette for overcoming a drawback associated with expansion and contraction of a flexible tube, and that the metering member is moved in accordance with the vertical movement of the pipette for overcoming a drawback associated with a relative positional change of the pipette and the metering member.

It is known that a detachable and disposable tip is used for preventing mutual contamination of specimens. For example, Japanese Unexamined Patent Publication No. 9-133686 (1997) discloses use of an electrically conductive tip for detection of a liquid surface.

Further, an analyzer is known which is adapted to analyze a specimen with the use of a disposable tip attached to a nozzle (for example, Japanese Unexamined Patent Publication No. 11-183484 (1999)).

However, the aforesaid arrangements are not satisfactory for more accurate sampling of a very small volume on the order of 2 μL, requiring further improvement. On the other hand, there is a demand for size reduction and cost reduction of such analyzers.

None of the aforesaid arrangements satisfy specification requirements.

In recent years, there has been a demand for point-of-care applications in the field of blood analysis to perform predetermined measuring operations beside a patient as required and immediately provide measurement results at the site. However, the conventional analyzers are not suitable for portable applications because the analyzers and reagent containers attached thereto are large in size.

Japanese Unexamined Patent Publication No. 11-183484 (1999), for example, discloses an analyzer adapted to analyze a specimen with the use of a disposable tip attached to a nozzle.

Japanese Unexamined Patent Publication No. 2-80937 (1990) discloses a particle analysis which is performed by sucking a liquid specimen into a probe and injecting the specimen into a flowcell (detector) with the probe being connected to an inlet of the flowcell.

However, these arrangements are not satisfactory in terms of the size reduction, requiring further improvement.

The present invention is directed to a liquid sampler which is capable of sampling a very small volume of liquid with an improved accuracy and allows for size reduction and cost reduction thereof.

The present invention is further directed to a blood analyzer which is suitable for the point-of-care applications and allows for size reduction and cost reduction thereof.

### SUMMARY OF THE INVENTION

In accordance with one aspect of the present invention, there is provided a liquid sampler which comprises: a metering pump including a cylinder having opposite end openings and a cylindrical cavity, a piston inserted in the cavity from one of the openings of the cylinder, and a driving source for reciprocally and linearly moving the piston; and a pipette directly connected to the other opening of the cylinder.

In accordance with another aspect of the present invention, there is provided a blood analyzer which comprises: the aforesaid liquid sampler; a driving mechanism for moving the liquid sampler horizontally and vertically; a liquid surface detecting section for detecting contact of a distal end of the pipette with a liquid surface; a controlling section for controlling the driving source and the driving mechanism upon reception of a signal from the liquid surface detecting section; a specimen vessel for containing a blood specimen; and an analyzing section for analyzing a test sample quantitatively dispensed out of the blood specimen from the specimen vessel by the liquid sampler.

### BRIEF DESCRIPTION OF THE DRAWINGS

FIG. 1 is a diagram illustrating the overall construction of a liquid sampling system employing a liquid sampler according to the present invention;

FIG. 2 is a table showing measurement results obtained by the inventive liquid sampler;

FIG. 3 is a table showing measurement results obtained by the inventive liquid sampler;

FIG. 4 is a front view of a simplified blood analyzer employing the inventive liquid sampler;

FIG. 5 is a diagram as viewed in an arrow direction A-A in FIG. 4;

FIG. 6 is a block diagram illustrating electric circuitry of the analyzer shown in FIG. 4;

FIG. 7 is a diagram illustrating how the analyzer of FIG. 4 performs a blood cell analysis; and

FIG. 8 is a plan view of a detection cassette of the analyzer of FIG. 4.

### DETAILED DESCRIPTION OF THE INVENTION

A liquid sampler according to the present invention comprises: a metering pump including a cylinder having opposite end openings and a cylindrical cavity, a piston inserted in the cavity from one of the openings of the cylinder, and a driving source for reciprocally and linearly moving the piston; and a pipette directly connected to the other opening of the cylinder.

In the inventive liquid sampler, the pipette, the cylinder and the driving source may be disposed in a coaxial relation.

The cylinder may have a channel extending from an outer circumference thereof to the cavity for supplying a cleaning liquid into the cavity.

The liquid sampler may further comprise an electromagnetic valve provided in the vicinity of the cylinder, and the electromagnetic valve may be adapted to control the supply of the cleaning liquid into the cavity via the channel.

The liquid sampler may further comprise a driving mechanism for moving the metering pump having the pipette in at least one-dimensional directions.

Alternatively, the liquid sampler may further comprise a driving mechanism for horizontally and vertically moving the metering pump having the pipette.

The term “pipette” herein means a tubular member capable of sucking and discharging a fluid, and a preferred example thereof is a stainless tube having a tapered distal end.

The driving source may comprise a stepping motor and a converting section for converting a rotational motion of the stepping motor into a linear motion and transmitting the linear motion to the piston.

The liquid sampler may further comprise a liquid surface detecting section for detecting contact of the distal end of the pipette with a liquid surface.

The pipette may be formed of an electrically conductive material, and the liquid surface detecting section may be adapted to detect the liquid surface on the basis of a change in impedance or electrostatic capacity between the pipette and the liquid surface. The liquid surface detecting section serves to minimize the area of the contact between the pipette and the liquid for improvement of the metering accuracy.

Where it is desirable to perfectly prevent the contamination of the pipette, an electrically conductive disposable pipette may be used as the pipette. The disposable pipette is held in intimate contact with the distal end of the cylinder.

A blood analyzer according to the present invention comprises: the aforesaid liquid sampler; a driving mechanism for moving the liquid sampler horizontally and vertically; a liquid surface detecting section for detecting contact of the distal end of the pipette with the liquid surface; a controlling section for controlling the pump driving source and the driving mechanism upon reception of a signal from the liquid surface detecting section; a specimen vessel for containing a blood specimen; and an analyzing section for analyzing a test sample quantitatively dispensed out of the blood specimen from the specimen vessel by the liquid sampler.

In the inventive blood analyzer, the analyzing section may comprise a detection member which includes a channel having an inlet and an outlet provided at opposite ends thereof and an orifice provided between the inlet and the outlet, and a detection section for detecting a change in impedance of the test sample when the test sample flows through the orifice. In this case, the controlling section may function to control the pump driving source and the driving mechanism so as to cause the metering pump to quantitatively suck the blood specimen from the specimen vessel via the pipette and quantitatively inject the sucked specimen as the test sample into the inlet of the detection member via the pipette.

The analyzing section may include a reagent vessel for containing a predetermined volume of a reagent, and the controlling section may function to control the pump driving source and the driving mechanism so as to cause the metering pump to quantitatively suck the blood specimen from the specimen vessel, quantitatively inject the sucked specimen into the reagent vessel to dilute the specimen, and quantitatively inject the diluted specimen as the test sample into the inlet of the detection member.

In this case, the controlling section calculates the number of red blood cells in the blood specimen on the basis of the change in the impedance detected by the detection section.

The analyzing section may include a reagent vessel for containing a predetermined volume of a reagent and a hemolyzing agent vessel for containing a hemolyzing agent, and the controlling section may function to control the pump driving source and the driving mechanism so as to cause the metering pump to quantitatively suck the blood specimen from the specimen vessel, quantitatively inject the sucked specimen into the reagent vessel to dilute the specimen, suck the hemolyzing agent from the hemolyzing agent vessel, inject the sucked hemolyzing agent into the reagent vessel to hemolyze the diluted specimen, and quantitatively inject the hemolyzed specimen as the test sample into the inlet of the detection member.

In this case, the controlling section calculates the number of white blood cells in the blood specimen on the basis of the change in the impedance detected by the detection section.

The analyzing section may include a reagent vessel for containing a predetermined volume of a reagent, a hemolyzing agent vessel for containing a hemolyzing agent and an absorbance measuring section for measuring the absorbance of a content in the reagent vessel, and the controlling section may function to control the pump driving source and the driving mechanism so as to cause the metering pump to quantitatively suck the blood specimen from the specimen vessel, quantitatively inject the sucked specimen into the reagent vessel to dilute the specimen, suck the hemolyzing agent from the hemolyzing agent vessel, and inject the sucked hemolyzing agent into the reagent vessel to hemolyze the diluted specimen.

In this case, the controlling section calculates the amount of hemoglobin in the blood specimen on the basis of the absorbance measured by the absorbance measuring section.

The absorbance measuring section may include a green LED for irradiating the content with light, and a photodiode for detecting light transmitted through the content.

The blood analyzer may further comprise a disposal section for collecting the pipette after the pipette is detached from the liquid sampler, and the controlling section may further function to control the driving mechanism so as to detach the pipette from the liquid sampler after use thereof and collect the detached pipette in the disposal section.

The blood analyzer may further comprise a pipette holder for holding a new disposable pipette, and the controlling section may further function to control the driving mechanism so as to attach the new disposable pipette in the pipette holder to the liquid sampler after the used pipette is detached from the liquid sampler.

Liquid Sampling System

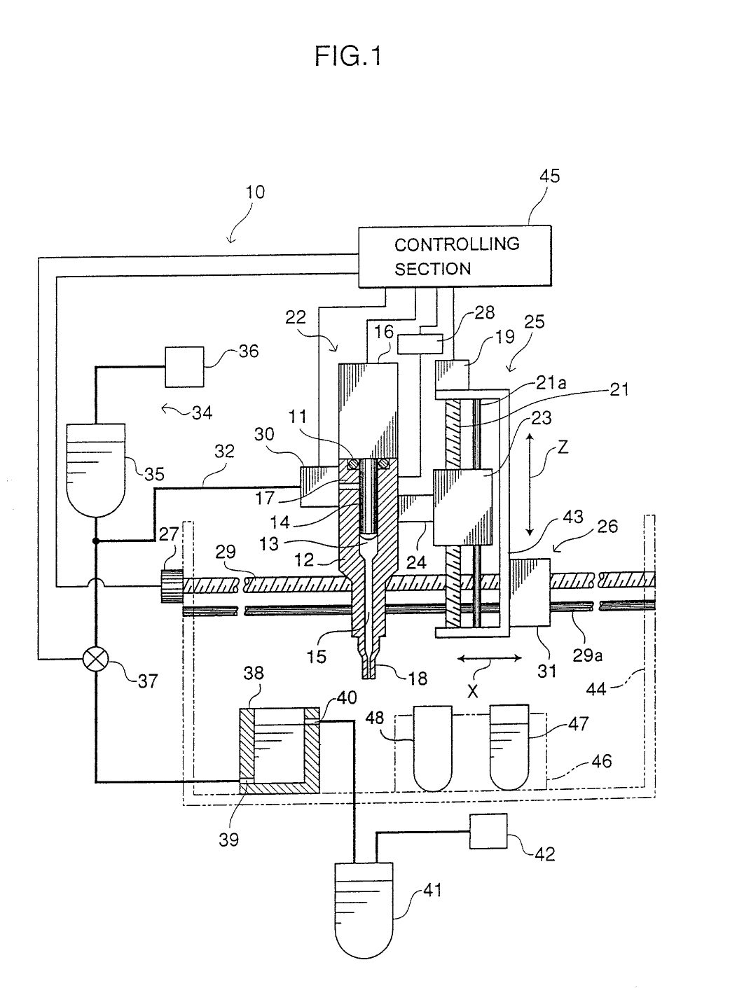

FIG. 1 is a diagram illustrating an exemplary construction of a liquid sampling system employing a liquid sampler according to the present invention. The liquid sampling system 10 includes a liquid sampler 22 having a cylinder 12, a piston 14, a linear actuator 16, a pipette 18 and an electromagnetic valve 30, which are unitarily provided. The liquid sampling system 10 further includes: a vertical driving mechanism 25 and a horizontal driving mechanism 26 adapted to hold the sampler 22 for moving the sampler 22 in arrow directions Z and X, respectively; a liquid surface detecting section 28 for detecting contact of a distal end of the pipette 18 with a liquid surface; a controlling section 45 for controlling operations of the actuator 16, the vertical and horizontal driving mechanisms 25, 26, and the like; and a cleaning liquid supplying section 34 connected via a tube to the valve 30 fixed to the cylinder 12.

The cylinder 12 has a cavity 13 coaxially formed therein, and two channels 15, 17 provided therein in communication with the cavity 13. The channel 15 communicates with the pipette 18 at a lower end of the cylinder 12, and the channel 17 communicates with an outlet port of the valve 30 at an upper side face of the cylinder 12. In this embodiment, the cylinder 12 and the pipette 18 are integrally formed of a stainless. Alternatively, the cylinder 12 and the pipette 18 may be formed as separate members which are directly connected to each other without the intervention of a flexible member. Where the cylinder 12 is formed of a resin and the pipette 18 is formed of a stainless, for example, the cylinder 12 and the pipette 18 may be bonded to each other with an adhesive or the like.

The linear actuator 16 is provided on an upper end of the cylinder 12 for driving the piston 14 of a ceramic (diameter: 3 mm).

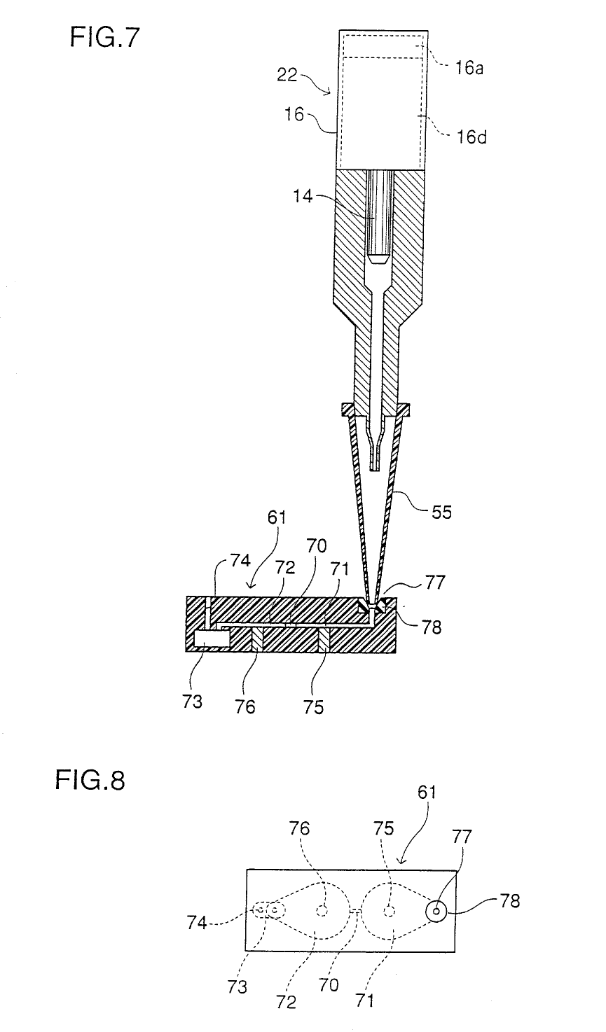

The linear actuator 16 incorporates a stepping motor 16a, and has a converting section 16b for converting a rotational motion of the stepping motor 16a in opposite directions into a reciprocal linear motion of a drive shaft of the linear actuator 16 (FIG. 7). The drive shaft of the linear actuator 16 is coupled to the piston 14 which is to be reciprocally and linearly moved in the cavity 13. While the piston 14 is moved within the cavity 13, a seal member (O-ring) 11 prevents liquid in the cavity 13 from leaking into the linear actuator 16. The drive shaft of the linear actuator 16 travels 0.00635 mm for each step of stepwise rotation of the stepping motor. More specifically, where the piston 14 of the liquid sampler 22 has an outer diameter of 3 mm, the liquid sampler 22 has a metering resolution of 0.0449 μL.

The sampler 22 is held by the vertical driving mechanism 25 via a holder 24 of an insulating material. The vertical driving mechanism 25 is mounted on the horizontal driving mechanism 26. Therefore, the sampler 22 is movable in the arrow directions Z and X.

In the vertical driving mechanism 25, a movable member 23 is slidably supported around a guide shaft 21a extending therethrough in engagement with a ball thread 21 extending parallel to the guide shaft 21a, and adapted to be moved in the arrow direction Z by rotation of a stepping motor 19. The guide shaft 21a, the ball thread 21 and the stepping motor 19 are fixed to a frame 43.

In the horizontal driving mechanism 26, a movable member 31 is slidably supported around a guide shaft 29a extending therethrough in engagement with a ball thread 29 extending parallel to the guide shaft 29a, and adapted to be moved in the arrow direction X by rotation of a stepping motor 27. The guide shaft 29a, the ball thread 29 and the stepping motor 27 are fixed to a metal frame 44. The frame 43 of the vertical driving mechanism 25 is fixed to the movable member 31. The frame 44 is electrically grounded.

The liquid surface detecting section 28 functions to detect the contact of the electrically conductive pipette 18 with the liquid surface. The detecting section 28 includes a known detector adapted to detect a change in electrostatic capacity between the pipette 18 and the frame 44 (ground) caused by the contact of the pipette 18 with the liquid surface, e.g., a detector adapted to detect a change in the amplitude of a high frequency voltage applied between the pipette 18 and the ground via a resistor.

The cleaning liquid supplying section 34 is connected to an inlet port of the valve 30 via the flexible tube 32. The cleaning liquid supplying section 34 includes a tank 35 for storing a cleaning liquid, and a positive pressure source 36 connected to the cleaning liquid tank.

The cleaning liquid is supplied into a cleaning vessel 38 for cleaning the pipette 18 from the cleaning liquid tank 35 via an electromagnetic valve 37 and a lower supply port 39. A negative pressure source (vacuum source) 42 is connected to a drain tank 41. The liquid in the cleaning vessel 38 is drained from an upper drain port 40 thereby to be collected in the drain tank 41.

An explanation will be given to a basic operation of the liquid sampling system 10 according to this embodiment. A detection signal of the liquid surface detecting section 28 is inputted to the controlling section 45, and the controlling section 45 controls the operations and operation timings of the valves 30, 37, the motors 19, 27, the linear actuator 16 and the like. As shown in FIG. 1, a specimen vessel 47 and an empty vessel 48 are set in a rack 46.

At an initial stage, the valves 30, 37 are closed. The sampler 22 is located in an upper initial position.

Upon actuation of the motors 19, 27, the sampler 22 is moved in the arrow direction X to a predetermined position, then moved down, and stopped at a predetermined height. The predetermined position is a position associated with the vessel 47, and the predetermined height is a level determined on the basis of the detection of the liquid surface. When the distal end of the pipette 18 is brought into contact with the surface of a liquid specimen in the vessel 47, the liquid surface detecting section 28 detects the contact, and applies a detection signal to the controlling section 45. The controlling section 45 controls the motor 19 so that the pipette 18 is further moved down by a distance corresponding to a volume of the liquid specimen to be sucked, and then stopped.

The linear actuator 16 is operated by a predetermined number of steps, whereby the piston 14 is moved up to cause the pipette 18 to suck the predetermined volume of the liquid specimen.

Subsequently, the motor 19 is rotated in a reverse direction, whereby the sampler 22 is moved up in the arrow direction Z to the initial height. By the rotation of the motor 27, the sampler 22 is moved in the arrow direction X, and stopped above the vessel 48. Then, the pipette 18 is moved down and stopped within the vessel 48 by the operation of the motor 19.

The linear actuator 16 is operated in a reverse direction by a predetermined number of steps to move down the piston 14, whereby the predetermined volume of the liquid specimen is discharged from the pipette 18 into the vessel 48.

Then, the pipette 18 is cleaned. More specifically, the pipette 18 is inserted into the cleaning vessel 38 on the basis of the detection of the liquid surface by driving the vertical and horizontal driving mechanisms 25, 26. With the valves 30, 37 being opened, the cleaning liquid is discharged from the pipette 18 and, at the same time, supplied from the lower supply port 39 of the cleaning vessel 38. Thus, the interior and exterior of the pipette 18 are cleaned. The liquid within the cleaning vessel 38 is drained from the drain port 40 thereby to be collected in the drain tank 41, so that the surface of the cleaning liquid in the cleaning vessel 38 is maintained substantially at a constant level.

Next, an explanation will be given to a metering accuracy determined on the basis of actual measurements.

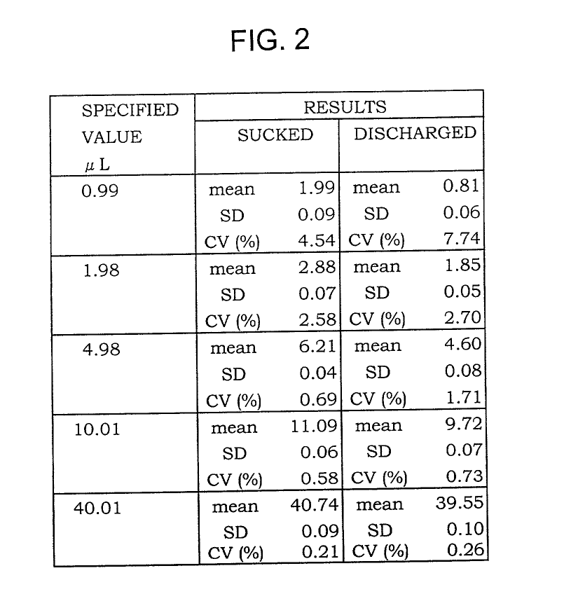

FIG. 2 is a table showing mean values, standard deviations SD and coefficients of variation CV calculated from actual measurements obtained with the use of the sampling system of this embodiment. The number of samples was n=20.

Sucked liquid volumes were each greater by 1 μL than a specified value, and discharged liquid volumes were each equal to the specified value. An electronic scale was used for the measurement. The reproducibility of the metering for each volume was satisfactory (particularly for smaller volumes). The sucked or discharged liquid volume y had a linear correlation with respect to the specified values x as expressed by y=0.9921×+1.0778 or by y=0.9935×−0.2118, respectively. For further improvement of the linearity accuracy, it is merely necessary to correct the driving amount of the linear actuator 16 on the basis of the linear correlation.

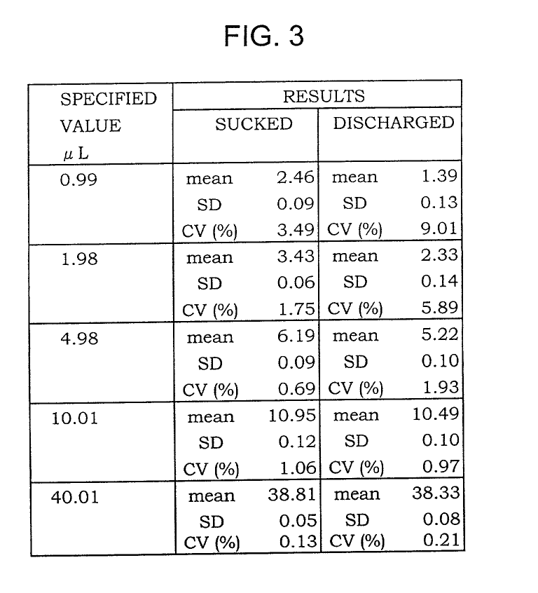

As will be described later, a disposable pipette may be used as the pipette 18. FIG. 3 is a table showing actual measurements obtained when the disposable pipette was used. The number of samples was n=20. Sucked liquid volumes were each greater by 2 μL than a specified value, and discharged liquid volumes were each greater by 1 μL than the specified value. The constant of variation CV for each volume was satisfactory (particularly for smaller volumes). The sucked or discharged liquid volumes y had a linear correlation with respect to the specified values x as expressed by y=0.9310×+1.5716 or by y=0.9456×−0.5912, respectively. The driving amount of the linear actuator 16 may be corrected in the aforesaid manner.

Blood Analyzer

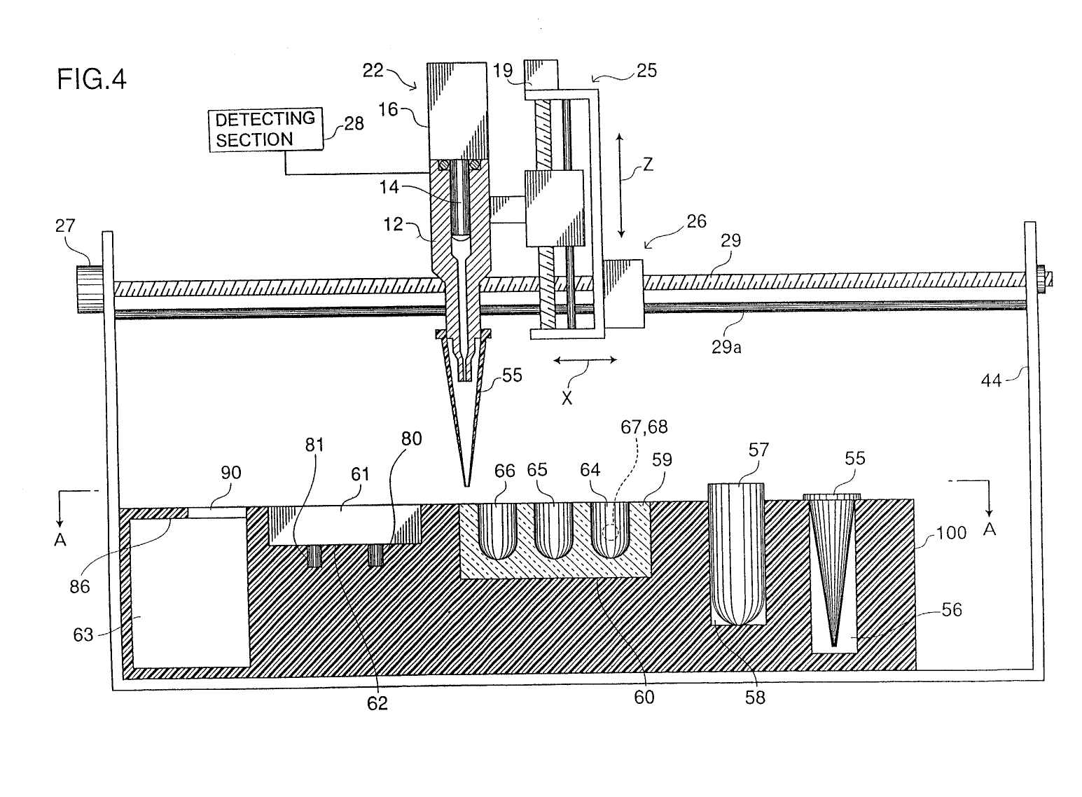

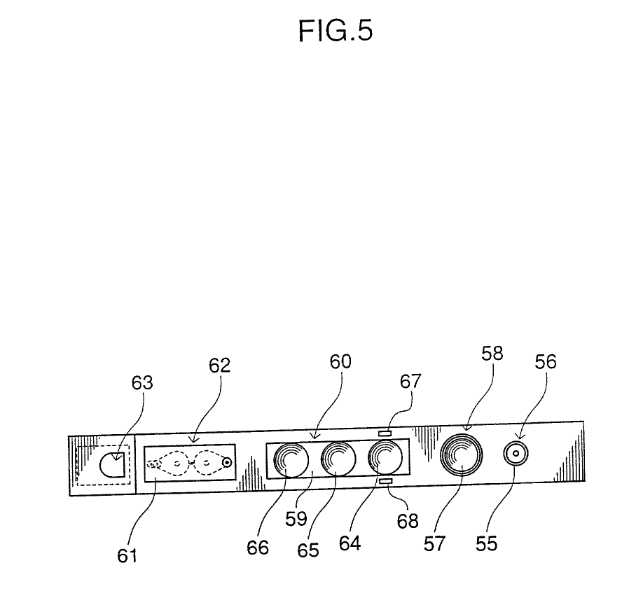

With reference to FIGS. 4 to 8, an explanation will be given to one example of a simplified blood analyzer which employs the liquid sampler 22 and the vertical and horizontal driving mechanisms 25, 26 shown in FIG. 1. FIG. 4 is a front view of the simplified blood analyzer. FIG. 5 is a diagram as viewed in an arrow direction A-A in FIG. 4. The simplified blood analyzer is adapted to perform a blood analysis by employing a disposable pipette (hereinafter referred to as “tip”) 55 detachably connected to the sampler 22 for quantitatively sucking and discharging a blood specimen. After completion of the analysis, the tip 55 is discarded, so that the tip 55 need not be cleaned. Therefore, the sampler 22 shown in FIG. 4 does not include the valve 30 nor the channel 17 shown in FIG. 1. The sampler has the pipette 18, which may be eliminated.

As shown in FIGS. 4 and 5, the simplified blood analyzer includes a rack 100 mounted on a frame 44, and the rack 100 has a tip holder 56 for holding the tip 55, a specimen vessel holder 58 for holding a specimen vessel 57, a reagent cassette holder 60 for holding a reagent cassette 59, a detection cassette holder 62 for holding a detection cassette 61, and a tip disposal section 63.

The tip 55 is formed of a mixture of a resin such as polypropylene and an electrically conductive fibers such as of black carbon and, therefore, has an electric conductivity.

A blood specimen obtained from a subject is contained in the specimen vessel 57.

The reagent cassette 59 is formed of a transparent material (e.g., glass), and has recesses 64, 65 for containing a 1000-μL diluent and a recess 66 for containing a 500-μL hemolyzing agent.

When the reagent cassette 59 is set in the reagent cassette holder 60, the recess 64 is located between a light emitting element 67 of a green LED and a light receiving element 68 of a photodiode incorporated in the rack 100 (FIG. 5).

The detection cassette 61 is molded from a polystyrene resin. As shown in FIGS. 7 and 8, the detection cassette 61 has a 120-ìm long fine hole 70 having a 100-ìm square cross section in an inner center portion thereof. Channels 71, 72 are provided on opposite sides of the fine hole 70 in communication with the fine hole 70. The channel 71 communicates with an inlet 77 provided on an upper face of the detection cassette 61, and the channel 72 communicates with a drain tank 73. The drain tank 73 is open to the atmosphere via a vent hole 74. A packing 78 is provided in the inlet 77. Rod electrodes 75, 76 of SUS316 are provided at the bottom of the cassette 61. Upper ends of the electrodes 75 and 76 are exposed to the channels 71 and 72, respectively, and lower ends of the electrodes 75, 76 are exposed to a lower face of the detection cassette 61. When the detection cassette 61 is set in the detection cassette holder 62, the electrodes 75 and 76 are brought into contact with contact terminals 80 and 81, respectively, provided in the rack 100 (FIG. 4) for electrical connection therebetween.

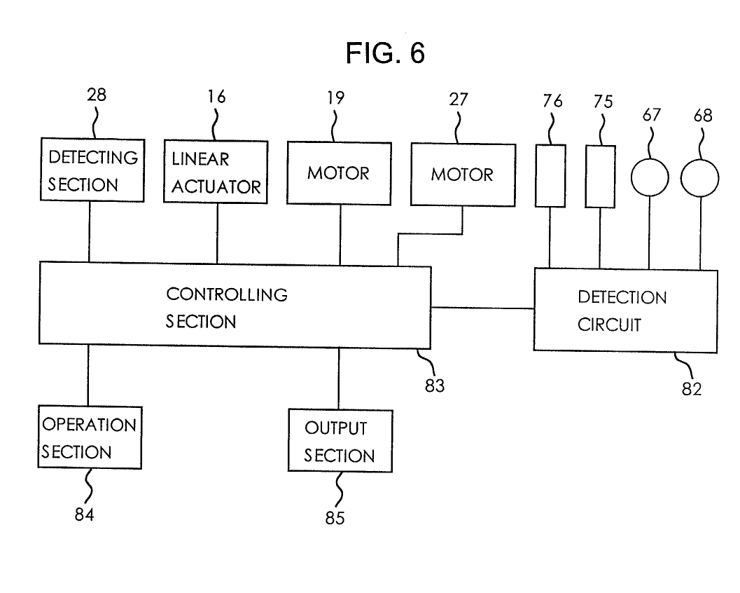

FIG. 6 is a block diagram illustrating electric circuitry of the simplified blood analyzer. A detection circuit 82 is adapted to detect signals from the light emitting element 67, the light receiving element 68 and the terminals 75, 76. The controlling section 83 receives signals outputted from the detection circuit 82, the liquid surface detecting section 28 and an operation section 84, and outputs signals to the linear actuator 16, the motors 19, 27 and an output section 85.

An explanation will be given to an operation to be performed by the blood analyzer.

Prior to the analysis, the tip 55, the specimen vessel 57, the reagent cassette 59 and the detection cassette 61 are set in the tip holder 56, the specimen vessel holder 58, the reagent cassette holder 60 and the detection cassette holder 62, respectively, of the rack 100 as shown in FIG. 4.

Upon depression of a start switch of the operation section 84, the controlling section 83 actuates the respective components.

First, the sampler 22 is moved down toward the tip holder 56 by the vertical driving mechanism 25 to insert the pipette 18 into a new tip 55 set in the tip holder 56, and moved up with the tip 55 fitted around a distal end portion of the cylinder 12 (in friction engagement therewith). On the other hand, the absorbance I₀ of the diluent in the recess 64 is measured by the light emitting element 67 and the light receiving element 68 (for blank measurement), and stored in the controlling section 83 after A/D-conversion thereof by the detection circuit 82.

The sampler 22 holding the tip 55 is moved to the upper side of the specimen vessel 57, and then moved down to bring the distal end of the tip 55 into contact with the surface of the blood specimen. The contact of the distal end of the tip 55 with the surface of the blood specimen is detected by the liquid surface detecting section 28. When the tip is inserted to a predetermined depth in the blood specimen, the controlling section 83 stops the vertical driving mechanism 25, and then drives the actuator 16 to suck a 2-μL aliquot of the blood specimen into the tip.

The sampler 22 discharges the sucked blood specimen into the 1000-μL diluent in the recess 64 for preparation of a primary test sample diluted about 500 times.

Then, a 10-μL aliquot of the primary test sample thus prepared is sucked into the tip and discharged into the recess A65 by the sampler 22 for preparation of a secondary test sample diluted about 50,000 times (for analysis of red blood cells).

The sampler 22 sucks a 100-μL aliquot of the thus prepared secondary test sample into the tip, and then is moved down toward the detection cassette 61, whereby the distal end of the tip 55 is brought into intimate press contact with the packing 78 of the inlet 77 as shown in FIG. 7.

Upon actuation of the actuator 16, the sucked secondary test sample is injected into the detection cassette 61 from the inlet 77. The secondary test sample fills the channel 71, the fine hole 70 and the channel 72, and then drained into the drain tank 73. While the secondary test sample flows through the fine hole 70 at a constant flow rate, blood cells pass through the fine hole 70. Electrical information based on a change in electrical resistance occurring at this time is sent to the detection circuit 82 via the electrodes 75, 76 and the terminals 80, 81. The detection circuit 82 detects pulse signals corresponding to the respective blood cells. The pulse signals are subjected to amplification and waveform modification, and then the number of the pulse signals is counted. The controlling section 83 calculates the number of red blood cells per 1-μL blood specimen on the basis of the number of the pulse signals counted during a period in which the predetermined volume of the secondary test sample flows through the fine hole.

Subsequently, the sampler 22 sucks a 200-μL aliquot of the hemolyzing agent from the recess 66 of the reagent cassette 59 into the tip 55, and then the distal end of the tip 55 is brought into intimate contact with the inlet 77 of the detection cassette 61. The 200-μL hemolyzing agent and then air are discharged from the tip 55 into the detection cassette 61 to clean the channel 71, the fine hole 70 and the channel 72, and the resulting drainage is collected in the drain tank 73.

Subsequently, a 30-μL aliquot of the hemolyzing agent is sucked into the tip 55 from the recess 66 of the reagent cassette 59 and discharged into the primary test sample in the recess 64 of the reagent cassette 59 by the sampler 22. Then, the sucking and discharging operations are repeated a plurality of times for hemolyzation of the primary test sample. Thus, a test sample for analysis of white blood cells and hemoglobin is prepared.

A 100-μL aliquot of the hemolyzed primary test sample is sucked into the tip 55 and, with the distal end of the tip 55 brought into intimate press contact with the packing 78 of the inlet 77, injected into the detection cassette 61 from the inlet 77 by the sampler 22. The detection circuit 82 counts the number of pulse signals, and the controlling section 83 calculates the number of white blood cells per 1-μL blood specimen.

On the other hand, the absorbance I₁ of the hemolyzed primary test sample is measured by the light emitting element 67 and the light receiving element 68 disposed on opposite sides of the recess 64 and, after A/D conversion thereof, the controlling section 83 determines the amount of hemoglobin by a known method on the basis of the absorbance I₁ and the blank absorbance I₀ previously measured.

The number of red blood cells, the number of white blood cells and the amount of hemoglobin thus calculated are displayed on an LCD display of the output section 85 or printed out by a printer of the output section 85.

Subsequently, the sampler 22 is moved to the upper side of the tip disposal section 63 and completely inserted into the tip disposal section 63 through an opening 90, and an upper end of the tip 55 is caught by a projection 86 for removal of the tip 55. Then, the sampler 22 returns to the initial position.

When the next specimen is to be analyzed, a new tip 55, a new specimen vessel 57 and a new reagent cassette 59 are set in the tip holder 56, the specimen vessel holder 58 and the reagent cassette holder 60, respectively, and then the start switch of the operation section 84 is depressed to start the analysis. After the analysis is performed a predetermined number of times, the detection cassette 61 is also replaced with a new one.

This embodiment is directed to analysis of red blood cells, white blood cells and hemoglobin. If only the hemoglobin is to be analyzed, there is no need to provide the detection cassette 61 nor to perform the secondary test sample preparing operation and the detection cassette cleaning operation.

Since the pipette and the metering pump are directly connected to each other in a unitary relation without the intervention of a tube in the liquid sampler according to the present invention, the metering accuracy can be improved without the expansion and contraction of the tube. Further, the unitary arrangement of the pipette and the metering pump prevents a change in the positional relationship between the pipette and the metering pump, thereby improving the metering accuracy. Moreover, the unitary arrangement allows for size reduction and cost reduction.

The pipette, the cylinder and the driving source are disposed in a coaxial relation. This allows for further improvement of the accuracy, further size reduction and further cost reduction.

A blood analyzer suitable for point-of-care applications can be provided by employing the smaller-scale liquid sampler according to the present invention in combination with a smaller-scale simplified analyzer.

What is claimed is:

1. A liquid sampler comprising:
a metering pump including a cylinder having opposite end openings and a cylindrical cavity, a piston inserted in the cavity from one of the openings of the cylinder, and a driving source for reciprocally and linearly moving the piston; and
a pipette directly connected to the other opening of the cylinder.

2. A liquid sampler as set forth in claim 1, wherein the pipette, the cylinder and the driving source are disposed in a coaxial relation.

3. A liquid sampler as set forth in claim 1, wherein the cylinder has a channel extending from an outer circumference thereof to the cavity for supplying a cleaning liquid into the cavity.

4. A liquid sampler as set forth in claim 3, further comprising an electromagnetic valve provided in the vicinity of the cylinder for controlling the supply of the cleaning liquid into the cavity via the channel.

5. A liquid sampler as set forth in claim 1, wherein the pipette is a disposable pipette which is directly connected to the other opening of the cylinder in a detachable manner.

6. A liquid sampler as set forth in claim 1, further comprising a driving mechanism for moving the metering pump having the pipette in at least one-dimensional directions.

7. A liquid sampler as set forth in claim 1, further comprising a driving mechanism for horizontally and vertically moving the metering pump having the pipette.

8. A liquid sampler as set forth in claim 1, wherein the driving source comprises a stepping motor, and a converting section for converting a rotational motion of the stepping motor into a linear motion and transmitting the linear motion to the piston.

9. A liquid sampler as set forth in claim 1, further comprising a liquid surface detecting section for detecting contact of a distal end of the pipette with a liquid surface.

10. A liquid sampler as set forth in claim 9,
wherein the pipette is composed of an electrically conductive material,
wherein the liquid surface detecting section detects the liquid surface on the basis of a change in electrostatic capacity between the pipette and the liquid surface.

11. A blood analyzer comprising:
a liquid sampler which comprises a metering pump including a cylinder having opposite end openings and a cylindrical cavity, a piston inserted in the cavity from one of the openings of the cylinder, and a driving source for reciprocally and linearly moving the piston, and a disposable pipette attached to the other opening of the cylinder in a detachable manner;
a driving mechanism for moving the liquid sampler horizontally and vertically;
a liquid surface detecting section for detecting contact of a distal end of the pipette with a liquid surface;
a controlling section for controlling the pump driving source and the driving mechanism upon reception of a signal from the liquid surface detecting section;
a specimen vessel for containing a blood specimen; and
an analyzing section for analyzing a test sample quantitatively dispensed out of the blood specimen from the specimen vessel by the liquid sampler.

12. A blood analyzer as set forth in claim 11, wherein the analyzing section comprises a detection member which includes a channel having an inlet and an outlet provided at opposite ends thereof and an orifice provided between the inlet and the outlet, and a detection section for detecting a change in impedance of the test sample when the test sample flows through the orifice.

13. A blood analyzer as set forth in claim 12, wherein the controlling section functions to control the pump driving source and the driving mechanism so as to cause the metering pump to quantitatively suck the blood specimen from the specimen vessel via the pipette, and quantitatively inject the sucked specimen as the test sample into the inlet of the detection member via the pipette.

14. A blood analyzer as set forth in claim 12,
wherein the analyzing section includes a reagent vessel for containing a predetermined volume of a reagent,
wherein the controlling section functions to control the pump driving source and the driving mechanism so as to cause the metering pump to quantitatively suck the blood specimen from the specimen vessel, quantitatively inject the sucked specimen into the reagent vessel to dilute the specimen, and quantitatively inject the diluted specimen as the test sample into the inlet of the detection member.

15. A blood analyzer as set forth in claim 14, wherein the controlling section calculates the number of red blood cells in the blood specimen on the basis of the change in the impedance detected by the detection section.

16. A blood analyzer as set forth in claim 12,
wherein the analyzing section includes a reagent vessel for containing a predetermined volume of a reagent and a hemolyzing agent vessel for containing a hemolyzing agent,
wherein the controlling section functions to control the pump driving source and the driving mechanism so as to cause the metering pump to quantitatively suck the blood specimen from the specimen vessel, quantitatively inject the sucked specimen into the reagent vessel to dilute the specimen, suck the hemolyzing agent from the hemolyzing agent vessel, inject the sucked hemolyzing agent into the reagent vessel to hemolyze the diluted specimen, and quantitatively inject the hemolyzed specimen as the test sample into the inlet of the detection member.

17. A blood analyzer as set forth in claim 16, wherein the controlling section calculates the number of white blood cells in the blood specimen on the basis of the change in the impedance detected by the detection section.

18. A blood analyzer as set forth in claim 11,
wherein the analyzing section includes a reagent vessel for containing a predetermined volume of a reagent, a hemolyzing agent vessel for containing a hemolyzing agent, and an absorbance measuring section for measuring the absorbance of a content in the reagent vessel,
wherein the controlling section functions to control the pump driving source and the driving mechanism so as to cause the metering pump to quantitatively suck the blood specimen from the specimen vessel, quantitatively inject the sucked specimen into the reagent vessel to dilute the specimen, suck the hemolyzing agent from the hemolyzing agent vessel, and inject the sucked hemolyzing agent into the reagent vessel to hemolyze the diluted specimen.

19. A blood analyzer as set forth in claim 18, wherein the controlling section calculates the amount of hemoglobin in the blood specimen on the basis of the absorbance measured by the absorbance measuring section.

20. A blood analyzer as set forth in claim 18, wherein the absorbance measuring section includes a green LED for irradiating the content with light, and a photodiode for detecting light transmitted through the content.

21. A blood analyzer as set forth in claim 11, further comprising a disposal section for collecting the pipette after the pipette is detached from the liquid sampler, wherein the controlling section further functions to control the driving mechanism so as to detach the pipette from the liquid sampler after use thereof and collect the detached pipette in the disposal section.

22. A blood analyzer as set forth in claim 21, further comprising a pipette holder for holding a new disposable pipette, wherein the controlling section further functions to control the driving mechanism so as to attach the new disposable pipette in the pipette holder to the liquid sampler after the used pipette is detached from the liquid sampler.
